(19) United States
(12) Patent Application Publication
(10) Pub. No.: US 2025/0349009 A1
(43) Pub. Date: Nov. 13, 2025

(54) REPAIRING ASSIST SYSTEM AND ASSESSMENT METHOD THEREOF

(71) Applicant: Astron Medtech Corporation, Wilmington, DE (US)

(72) Inventors: Yi-Hsi HUANG, Taipei City (TW); I-Lin TSAI, Taipei City (TW); Kuei-Hua CHEN, Taipei City (TW)

(21) Appl. No.: 18/658,770

(22) Filed: May 8, 2024

(51) Int. Cl. G06T 7/00; A61B 90/00

(52) U.S. Cl. CPC G06T 7/0016; A61B 90/39; G06T 2207/10116; G06T 2207/10132

(57) ABSTRACT

A repairing assist system has a detection equipment having a cross-section detection device to capture multiple cross-section image to form an image group which includes at least one injury image and multiple healthy images. A guiding module is detachably assembled with the cross-section detection device and is applied to assist in marking an operation coordinate on a surface of an injury area after a strong center zone is determined.

### FIELD OF INVENTION

The present invention relates to an assist system, especially to a repairing assist system for tendons or ligaments and an assessment method thereof.

### BACKGROUND OF THE INVENTION

Tendon injuries (especially the Achilles or rotator tendons) is a common injury caused by work habits or sports injuries in modern life. Fixing an injured area with a medical brace or a cast to allow the injured area to repair itself is a common conservative treatment method. However, this treatment method often requires a long period of rest for the injured area to fully recover. If the tendon in the injured area is severely ruptured, the tendon end of the injured area will usually contract, which may form a 1-2 cm gap that a tendon repair surgery will be recommended.

Because a cross-sectional diameter of the tendon is small, the tendons are located deep in the body, and the injured area of the tendon is usually irregular, the end of the tendon rupture does not have a complete cross-section. Thus, during the surgery, a skin adjacent to the injured area is always turned up to directly confirm the ruptured part of the tendon and to decide the best suture point at the end of the tendons at two side of the gap. However, the large wounds caused after surgery are not only unsightly, but also cause a burden of care. Thus, it is urge to develop a repairing system which is minimally invasive and can accurately identify the damaged of the tendon and provide an optimal suture point during the surgery in the related field.

### SUMMARY OF THE INVENTION

In order to develop a minimally invasive repairing system the present invention provides a repairing assist system which can accurately identify the damaged and provide an optimal suture point. The repairing assist system comprises a detection equipment comprising a cross-section detection device which captures multiple cross-section image along a detection direction in real-time imaging, and forms an image group; the image group which comprises at least one injury image and multiple healthy images.

Wherein at least two healthy images are respectively arranged in front of and behind the least one injury image along the detection direction; and two strong center zones are respectively defined on the at least two healthy images; and a guiding module which is detachably assembled with the cross-section detection device, and is applied to assist in marking an operation coordinate on a surface of an injury area corresponding to the strong center zone.

The present invention further provides an assessment method of a repairing assist system has steps comprising:
obtaining an image group from a cross-section detection device imaging along a detection direction, wherein the image group comprises at least one injury image and multiple healthy images;
identifying one of the at least one injury image as a reference plane, and selecting at least two healthy images which are respectively arranged in front of and behind one of the at least one injury image along the detection direction;
analyzing at least two healthy images by scoping an outline which is corresponding to a cross-sectional edge of each at least two healthy images;
defining a centroid point according to the outline;
forming multiple connecting lines by connecting the centroid point to the outline radially;
defining multiple strong sites on each connecting line; and
forming a strong center zone by connecting each adjacent strong site.

The strong center zone can be used as an optimal suture point and be considered a candidate site for needle insertion during a repairing surgery to allow a surgical needle to pass through and guiding a surgical suture to be inserted. In addition, the strong center zone also provides sufficient strength to support a pulling force generated by a surgical suture during suturing.

Wherein the repairing assist system can be applied to a repairing surgery of an injured ankle ligament. Multiple bones of an ankle joint are connected each other through multiple ankle ligaments which are extend in different directions respectively. The detection equipment can apply the ultrasonic technology or a computed tomography (CT) technology to identify the injury site A′ of the injured ankle ligament and determined the strong center zone.

Wherein the repairing assist system can be applied to a repairing surgery of an injured shoulder tendon. A scapula is connected to multiple muscle groups extending in different directions through multiple shoulder tendons extending in different directions, allowing a shoulder/the scapula to rotate at multiple angles. Since the multiple shoulder tendons are densely arranged and partially overlap to each other, in order to avoid damaging other nearby healthy shoulder tendons, it is important to confirm the location of the injury site of the injured shoulder tendon. The detection equipment can apply the ultrasonic technology and use a humerus as a starting point to confirm the location of injury site of the injured shoulder tendon and obtain the coordinate of the strong center zone according to the position of injury site, for example: when the cross-section detection device moves toward the upper end of the scapula, cross-section images of a supraspinatus tendon can be captured; when the cross-section detection device moves toward the chest, cross-section images of a infraspinatus tendon can be captured.

Wherein the repairing assist system can be applied to a repairing surgery of an injured tendon of a thigh. A knee joint of the thigh connects to multiple muscle groups extending in different directions through multiple tendons extending in different directions, allowing the thigh to exhibit multi-angle movements. The detection equipment can apply the ultrasonic technology and use the knee joint as a starting point to confirm the location of injury site of the injured tendon and obtain the coordinate of the strong center zone according to the position of injury site, for example: when the cross-section detection device moving toward a groin, the cross-section images of a vastus lateralis, a rectus femoris, or a vastus medialis can be captured sequentially from the outside of the thigh to the inside of the thigh.

The repairing assist system the present invention provided can immediately perform the repairing surgery after the strong center zone is determined through the combination of detection equipment and the needle guide module. Since the detection equipment has the real-time imaging feature and the function of identifying the strong center zone, it can simultaneously confirm is the surgical coordinate or surgical device is corresponded to the strong center zone while operating.

The repairing assist system the present invention provided can avoid a large wound be made on the injury area when during repairing surgery, achieve the function of fixing and repairing the injured tendons of the injury site, and prevent a secondary injury formed and helping to accelerate the healing of injury area.

The repairing assist system combines the detection equipment and needle guide module when using, which achieve the effect of convenient positioning, marking and performing surgery during the operation.

### BRIEF DESCRIPTION OF THE DRAWINGS

FIG. 1 is a simulation scenario schematic diagram of a preferred embodiment of a system in accordance with the present invention;

FIG. 2A is a partially enlarged simulation perspective view of the system in FIG. 1;

FIG. 2B is a partially enlarged simulation top view of the system in FIG. 1;

FIG. 3 is a first simulation operate scenario perspective view of a preferred embodiment of a system in accordance with the present invention;

FIG. 4 is a second simulation operate scenario perspective view of a preferred embodiment of a system in accordance with the present invention;

FIG. 5 is a third simulation operate scenario top view of a preferred embodiment of a system in accordance with the present invention;

FIG. 6 is a fourth simulation operate scenario schematic diagram of a preferred embodiment of a system in accordance with the present invention;

FIGS. 7A-7D show ultrasound images of a damaged tendon according to the preferred embodiment of the present invention.

### DETAILED DESCRIPTION OF THE PREFERRED EMBODIMENTS

In order to make purposes, technical solutions, and advantages of the present invention to be clearer, the following content provides some preferred embodiments in accordance with the present invention.

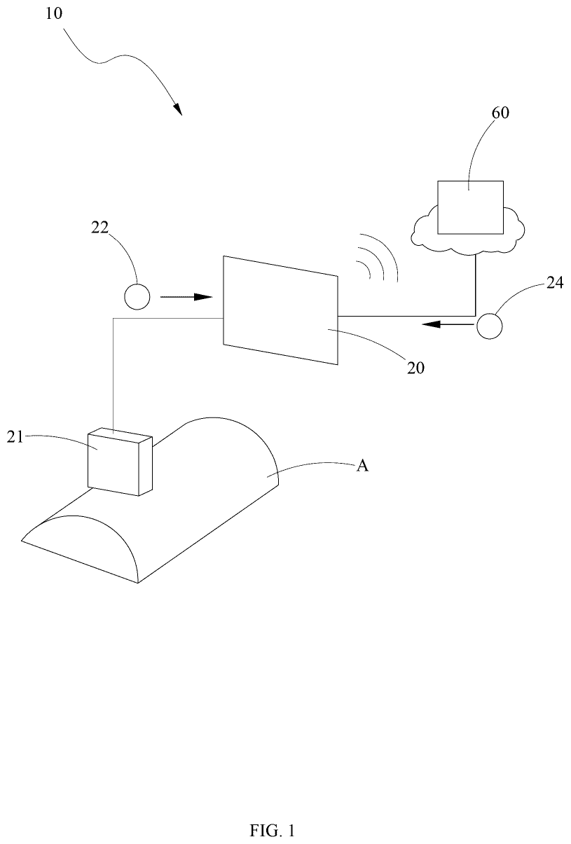

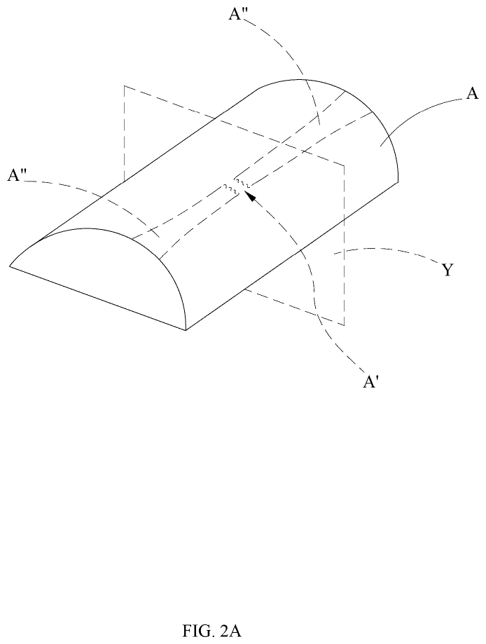

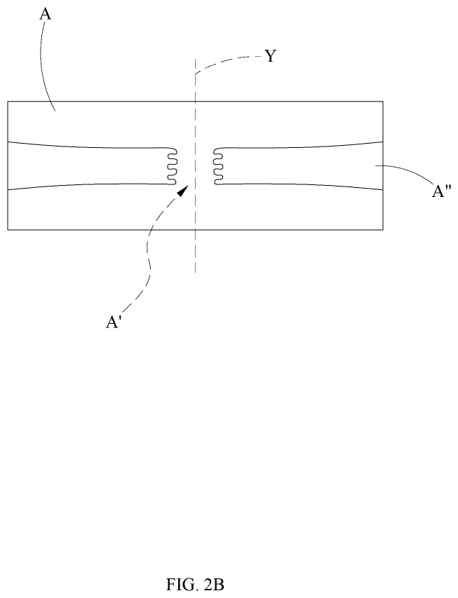

With reference to FIG. 1 to FIG. 3, a repairing assist system 10 in accordance with the present invention is provided, which is applied to assist in evaluating a recommended operating site before a repairing surgery, and assist in confirming the operating site when surgery. The repairing assist system 10 comprises a detection equipment 20 and a guiding module 30. The detection equipment 20 comprises a cross-section detection device 21 which can perform real-time imaging and capture multiple cross-section images along a detection direction on a surface of an injury area A to obtains an image group 22. The cross-section detection device 21 defines an image plane A1 when detecting.

The interior of the injury area A comprises an injury site A′ and two healthy part A″ adjacent to both sides of the injury site A′. The cross-section detection device 21 captures multiple cross-section image along the arrangement of the injury site A′ and two healthy part A″, and the image group 22 comprise at least one injury image corresponding to the injury site A′, and at least two healthy images corresponding to the two healthy part A″ respectively.

The image group 22 can be imaged on a screen in real time as the cross-section detection device 21 moves along the surface of the injury area A, and at least one injury image and at least two healthy images can be captured and stored in sequence according to a displacement direction of the cross-section detection device 21.

The said injury area A can be caused by a damage of a tendon, a ligament or a muscle due to rupture or inflammation, such as the Achilles tendon, rotators, deltoid or quadriceps muscles, etc.

The said detection equipment 20 is not limited in the present invention, it can be implemented by an ultrasound technology, an X-ray technology, a tomography technology or other medical imaging technology. A suitable medical imaging technology of the detection equipment 20 can be chosen according to the shape of type of the injury area A.

The present invention further uses an injury tendon as the injury area A as an example to illustrate an assessment method of the repairing assist system 10. Since the tendon is a soft tissue, the detection equipment 20 is implemented by the ultrasound technology.

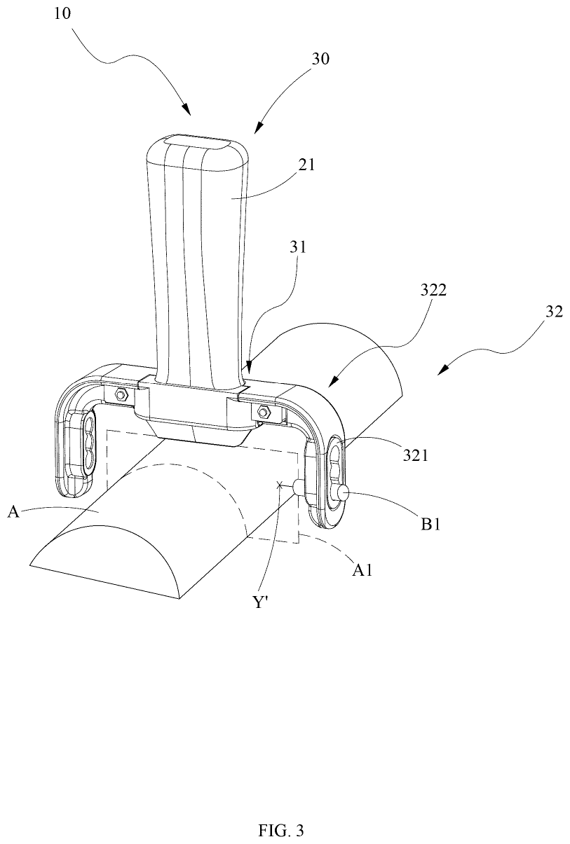

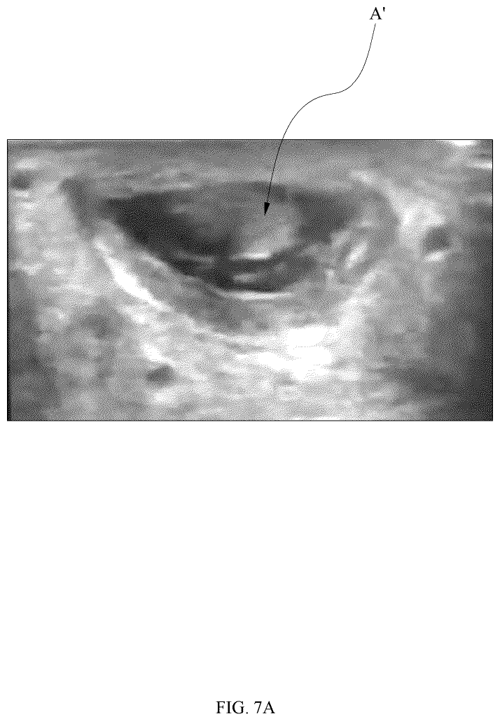

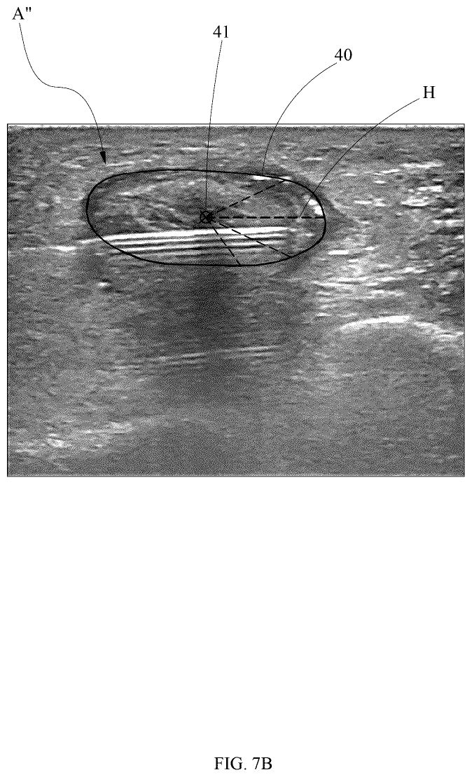

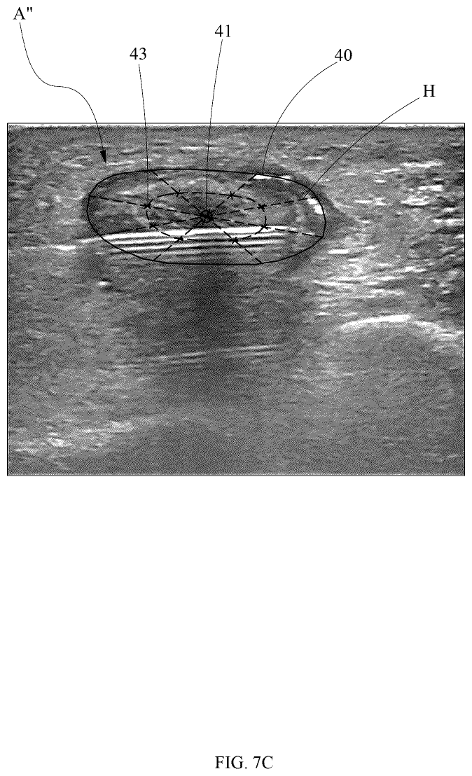

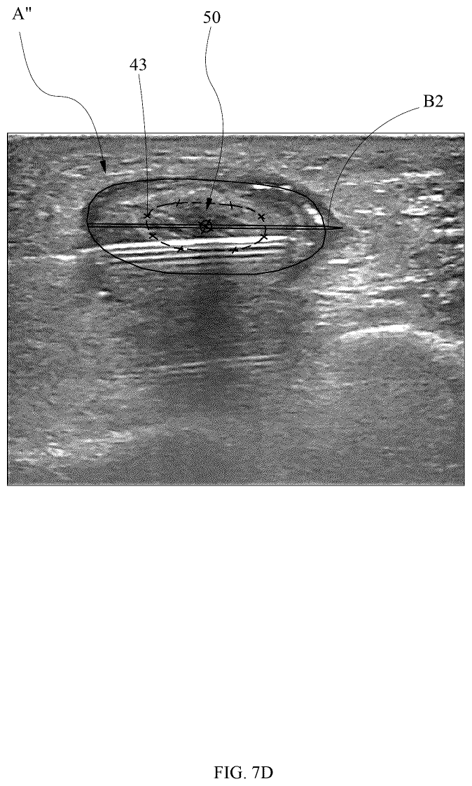

With reference to FIGS. 3 to 4 and FIG. 7A to 7D. FIG. 7A is a cross-section image corresponding to the injury site A′ of the injury tendon, showing a gap formed by the rupture of the tendon and an incomplete cross-sectional of the tendon. FIG. 7B is one of the cross-section images corresponding to the healthy part A″ and shows the complete tendon cross-section. The assessment method has the steps including:

S1, identifying a reference plane Y, and selecting two healthy images at two sides of the reference plane Y respectively:

After obtained the image group 22, using the cross-section images to identify the injury site A, and defining the injury image as the reference plane Y, then selecting two healthy images at two sides of the reference plane Y respectively (FIG. 7B).

S2, analyzing two healthy images:

As shown in FIG. 7B, an outline 40 is respectively defined at the two selected healthy images, which is corresponding to the edge of cross-sectional of the tendon in the healthy part A″, and a centroid point 41 can be define according to the outline 40.

S3, defining a strong center zone 50:

Referring to FIG. 7C and FIG. 7D, multiple connecting lines H are formed by radially connecting the centroid point 41 to the outline 40. Multiple strong sites 43 are defined on each connecting line H, wherein a distance between each strong site 43 and the centroid point 41 is less than 30 to 50 percent of a length of each connecting lines H. And the strong center zone 50 is formed in an area by connecting each adjacent strong site 43.

S4 (optional), marking the strong center zone 50:

After the strong center zone 50 is determined, detection equipment 20 can further mark the strong center zone 50 on each healthy image, and provides a coordinate corresponding to the strong center zone 50. The method for marking the strong center zone 50 is not limited, for example, the health center 50 can be directly real-time imaging combined with the image group 22 by labeling a pattern or a line. Thus, the strong center zone 50 can be quickly defined and clearly marked on corresponding cross-section images after the cross-section detection device 21 is moved.

Preferably, the coordinate can be determined by using the reference plane Y as a basal coordinate. For example, the coordinate is a three-dimensional coordinate corresponding to a length distance from the reference plane Y, a left/right distance from the centroid point 41 of the reference plane Y, and an up/down distance from the centroid point 41 of the reference plane Y.

More preferably, considering the reference plane Y and corresponding to the injury site A′, a complete cross-sectional shape may be inaccurate to define the centroid point 41. The left/right distance and the up/down distance of the three-dimensional coordinate also can be defined from the centroid point 41 of the healthy image

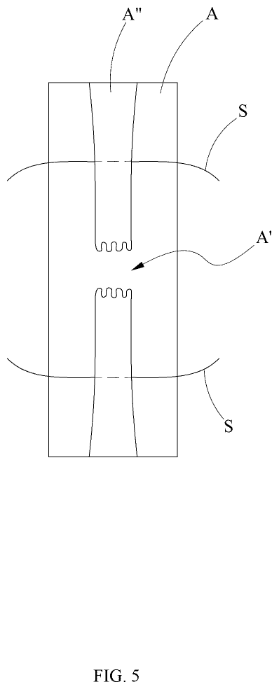

As the detection equipment 20 confirmed the strong center zone 50, the strong center zone 50 can be used as an optimal suture point and be considered a candidate site for needle insertion during a repairing surgery. Refers to FIG. 5 and FIG. 7D, the strong center zone 50 of the healthy part A″ is applied to ensure that the healthy part A″ can provide a sufficient range to allow a surgical needle B2 to pass through and guiding a surgical suture S to be inserted. In addition, the strong center zone 50 also provides a sufficient strength to support a pulling force generated by a surgical suture S for closing the gap formed the injury site A′ during suturing.

Preferably, an expected distance or an expected range corresponding to both sides of the reference plane Y can be preset when the detection equipment 20 is performed to form the image group 22. In other words, the detection equipment 20 can obtain at least one healthy tissue image corresponding to the expected distance or the expected range after the reference plane Y is identified, and then choose two healthy images at two sides of the reference plane Y that the strong center zone 50 can be determined.

More preferably, the detection equipment 20 can be connected to a learning database 60 through the network. The learning database 60 saves multiple injury images and multiple surgical records corresponding to multiple past surgeries. The detection equipment 20 performs deep learning based on the learning database 60 and generates an interpretation module 24. When repairing assist system 10 in use, the detection equipment 20 can interpret the cross-section images of the image group 22 according to the interpretation module 24, and can identify the injury site A′, a level of damage, and can provide a surgical advice. Said the surgical advice may be to select the healthy images at two sides of the reference plane Y by directly confirm the expected distance or the expected range, and directly determine the strong center zone 50.

The purpose of the expected distance/range mentioned above is to prevent the detection equipment 20 from capturing unnecessary images of the healthy tissue to affect an identification result of the strong center zone 50 by pre-setting the expected distance/range which the strong center zone 50 may be include in, and prevent unnecessary waste of time.

In some embodiments, the repairing assist system 10 can be applied to a repairing surgery of an injured ankle ligament. Multiple bones of an ankle joint are connected each other through multiple ankle ligaments which extend in different directions respectively. The detection equipment 20 can apply the ultrasonic technology or a computed tomography (CT) technology to identify the injury site A′ of the injured ankle ligament and determined the strong center zone 50.

In some embodiments, the repairing assist system 10 can be applied to a repairing surgery of an injured shoulder tendon. A scapula is connected to multiple muscle groups extending in different directions through multiple shoulder tendons extending in different directions, allowing a shoulder/the scapula to rotate at multiple angles. Since the multiple shoulder tendons are densely arranged and partially overlapped with each other, in order to avoid damaging other nearby healthy shoulder tendons, it is important to confirm the location of the injury site A′ of the injured shoulder tendon. The detection equipment 20 can apply the ultrasonic technology and use a humerus as a starting point to confirm the location of injury site A′ of the injured shoulder tendon and to obtain the coordinate of the strong center zone 50 according to the position of injury site A′. For example: when the cross-section detection device 21 moves toward the upper end of the scapula, cross-section images of a supraspinatus tendon can be captured. When the cross-section detection device 21 moves toward the chest, cross-section images of an infraspinatus tendon can be captured.

In other embodiment, the repairing assist system 10 can be applied to a repairing surgery of an injured tendon of a thigh. A knee joint of the thigh connects to multiple muscle groups extending in different directions through multiple tendons extending in different directions to allow the thigh to exhibit multi-angle movements. The detection equipment 20 can apply the ultrasonic technology and use the knee joint as a starting point to confirm the location of injury site A′ of the injured tendon and to obtain the coordinate of the strong center zone 50 according to the position of injury site A′. For example: when the cross-section detection device 21 moves toward a groin, the cross-section images of a vastus lateralis, a rectus femoris, or a vastus medialis can be captured sequentially from the outside of the thigh to the inside of the thigh.

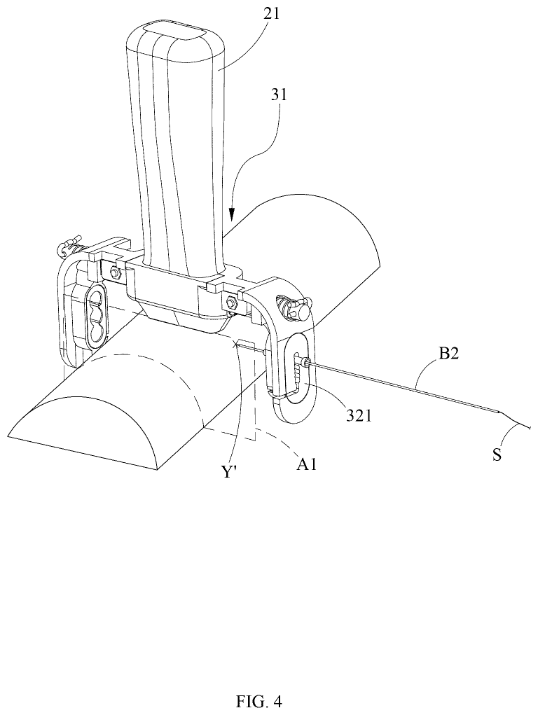

With reference to FIGS. 3 to 4. The guiding module 30 is applied to assist in marking an operation coordinate Y′ which is corresponding to the strong center zone 50 on the surface of the injury area A, and guiding the surgical needle B2 to pass through the injury area A from the operation coordinate Y′.

The guiding module 30 comprises an assembly portion 31 and at least one operating portion 32. The operating portion 32 is connected to at least a part of the assembly portion 31 and an operating space 33 is formed between the assembly portion 31 and the operating portion 32 to accommodate the injury area A in the operating space 33.

The assembly portion 31 is used for a detachable assembly in connection with cross-section detection device 21 so that the irradiating end of the cross-section detection device 21 corresponds to the operating space 33. When the cross-section detection device 21 is irradiated to the operating space 33, the cross-section images of the image group 22 are formed corresponding to the image plane A1. The method of assembling the assembly portion 31 and the cross-section detection device 21 is not limited. The assembly portion 31 may be a fixture for holding the cross-section detection device 21, or it may mutually snap with at least a part of the cross-section detection device 21. Thus, the detection equipment 20 may be combined with the guiding module 30 and may obtain the image group 22 when using guiding module 30.

The operating portion 32 comprises at least one module 321 and a module assembly structure 322 for detachably assembling the at least one module 321 in in the module assembly structure 322. The module 321 is detachably assembled with a surgical device. When the module 321 is assembled with the surgical device and disposed in the module assembly structure 122, an operating end of the surgical device aligns horizontally with the image plane A1. Wherein the surgical device can be a laser calibrator B1 for marking the operation coordinate Y′, or the surgical needle B2, with the surgical suture S there behind, for bring the surgical suture S to pass through the injury area A.

In the embodiment for the operating portion 32 provided by the present invention. When the assembly portion 31 is combined with cross-section detection device 21 and capturing the cross-section images, the surgical device can be the laser calibrator B1 which is adjustably held by at least one module 321. A laser end of the laser calibrator B1 is aligning with the image plane A1.

As two strong center zones 50 of selected healthy part A″ are defined, the image plane A1 of the cross-section detection device 21 is real-timing overlapped with the healthy image of the selected healthy part A″ which comprises one of the strong center zones 50. Combining the real-time imaging function of the detection equipment 20 and the characteristics of a laser light penetrating tissue, it can be confirmed if the laser light projected by the laser calibrator B1 is penetrated the strong center zones 50 on the healthy images of the selected healthy part A, and the position of strong center zone 50 can be determined by marking the operation coordinate Y′ on the surface of the injury area A.

When using the surgical needle B2 to bring the surgical suture S to pass through the injury area A. the surgical needle B2 can penetrate the affected part surface A and pass through the surface of the injury area A from the surgical coordinate Y′, at the meantime, it can be confirmed on healthy image if the surgical needle B2 has entered the strong center zone 50 through real-time imaging of the detection equipment 20 (FIG. 7D).

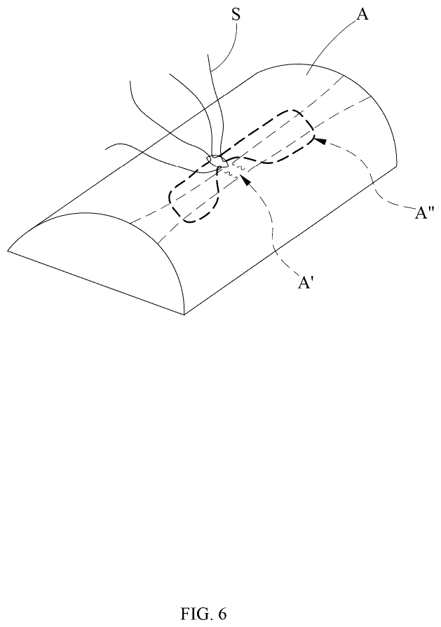

With reference to FIGS. 5 and 6, when performing repairing surgery, both strong center zones 50 at the two side of the injury site A′ are penetrated the surgical sutures S, and a surgical wound is opened on the surface injury area A between the two surgical sutures S. Then the two ends of the two surgical sutures S can be guided to the surgical wound that the gap of injury area A (the injured tendons) can be closed by binding the surgical sutures S located on two sides of the injury area A at the surgical wound, then the repairing surgery is completed.

The repairing assist system 10 in accordance with the present invention can immediately performs the repairing surgery after the strong center zone 50 is determined through the combination of detection equipment 20 and the needle guide module 30. Since the detection equipment 20 has the real-time imaging feature and the function of identifying the strong center zone 50, the surgical coordinate Y′ or surgical device corresponding s to the strong center zone 50 while operating can be simultaneously confirmed.

The repairing assist system 10 in accordance with the present invention can avoid a large wound be made on the injury area A when during repairing surgery to achieve the function of fixing and repairing the injured tendons of the injury site A′ and to prevent a secondary injury formed so as to accelerate the healing of injury area A.

The repairing assist system 10 is combined with the detection equipment 20 and needle guide module 30 when in use and achieves the effect of convenient positioning, marking and performing surgery during the operation.

What is claimed is:

1. A repairing assist system comprising:
a detection equipment comprising a cross-section detection device which captures multiple cross-section image along a detection direction in real-time imaging and forms an image group; the image group which comprises at least one injury image and multiple healthy images, wherein at least two healthy images are respectively arranged in a front of and a behind of the least one injury image along the detection direction; and two strong center zones are respectively defined on the at least two healthy images; and
a guiding module which is detachably assembled with the cross-section detection device, and is applied to assist in marking an operation coordinate on a surface of an injury area corresponding to the strong center zone.

2. The repairing assist system as claimed in claim 1, wherein the detection equipment is implemented by an ultrasound technology, an X-ray technology, a tomography technology or other medical imaging technology.

3. The repairing assist system as claimed in claim 1, wherein the detection equipment defines at least one injury image as a reference plane, and obtains multiple healthy images according to an expected distance or range after the reference plane is identified when the cross-section detection device is capturing.

4. The repairing assist system as claimed in claim 1, wherein the detection equipment further comprises a learning database which saves multiple historical injury images and multiple surgical records, wherein
the detection equipment performs a deep learning program based on the learning database and generates an interpretation module, and
the detection equipment identifies a location of body and a level of damage of the injury image according to the interpretation module and provides a surgical advice.

5. The repairing assist system as claimed in claim 1, wherein the image group is imaged on a screen in real time as the cross-section detection device is capturing.

6. The repairing assist system as claimed in claim 1, the at least one injury image shows damage of a tendon, a ligament or a muscle.

7. The repairing assist system as claimed in claim 1, the guiding module further comprising an assembly portion and at least one operating portion connected to at least a part of the assembly portion where an end of the at least one operating portion extends in the direction of an axis, an operating space is formed between the assembly portion, the operating space is applied to accommodate the injury area, wherein an irradiating end of the cross-section detection device extends into the operating space when the cross-section detection device is assembled with the guiding module, and the irradiating end defined an image plane, and the multiple cross-section images of the image group are formed corresponding to the image plane.

8. The repairing assist system as claimed in claim 7, wherein the operating portion is detachably assembled with a surgical device, an operating end of the surgical device aligns horizontally with the image plane when the surgical device assembled with the operating portion.

9. The repairing assist system as claimed in claim 8, wherein the surgical device is a laser calibrator for marking the operation coordinate, or a surgical needle with a surgical suture for bringing the surgical suture to pass through the injury area.

10. An assessment method of a repairing assist system has steps comprising:
obtaining an image group from a cross-section detection device imaging along a detection direction, wherein the image group comprises at least one injury image and multiple healthy images;
identifying one of the at least one injury image as a reference plane, and selecting at least two healthy images which are respectively arranged in a front of and a behind of one of the at least one injury image along the detection direction;
analyzing at least two healthy images by scoping an outline corresponding to a cross-sectional edge of each at least two healthy images;
defining a centroid point according to the outline;
forming multiple connecting lines by connecting the centroid point to the outline radially;
defining multiple strong sites on each connecting line; and
forming a strong center zone by connecting each adjacent strong site.

11. The assessment method as claimed in claim 10, wherein a distance between each strong site and the centroid point is less than 30 to 50 percent of a length of each connecting line.

12. The assessment method as claimed in claim 11, wherein marking the strong center zone on each of the at least two healthy images after the strong center zone is determined, and forming a coordinate of the strong center zone.

13. The assessment method as claimed in claim 12, wherein the coordinate is a three-dimensional coordinate.

14. The assessment method as claimed in claim 12, the coordinate is determined by using the reference plane Y as a basal coordinate.

15. The assessment method as claimed in claim 12, the coordinate is determined by using a relative distance from the centroid point.
